(19) United States
(12) Patent Application Publication
(10) Pub. No.: US 2014/0276198 A1
(43) Pub. Date: Sep. 18, 2014

(54) PRESSURE SENSING CATHETER

(71) Applicant: Cook Medical Technologies LLC, Bloomington, IN (US)

(72) Inventors: Ronak Dunung, Bloomington, IN (US); Jimmy Taylor, Poland, IN (US)

(73) Assignee: Cook Medical Technologies LLC, Bloomington, IN (US)

(21) Appl. No.: 14/196,493

(22) Filed: Mar. 4, 2014

Related U.S. Application Data
(60) Provisional application No. 61/786,866, filed on Mar. 15, 2013.

(51) Int. Cl. A61B 5/03; A61B 5/00

(52) U.S. Cl. CPC A61B 5/036; A61B 5/6852
USPC 600/561

(57) ABSTRACT

A pressure sensing catheter may include an elongate tubular catheter body having a first lumen extending longitudinally within the catheter body, and a second lumen extending longitudinally within the catheter body adjacent to the first lumen. Each of the first and second lumens may include proximal and distal end openings positioned at proximal and distal ends, respectively, of the catheter body. A tip portion may extend distally from the distal end of the catheter body and may include an outer wall and a tip lumen. The tip lumen may include a first and second end openings coupled to the distal end openings of the first and second lumens, respectively. The first lumen and the second lumen may be in fluid communication with one another through the tip lumen. The tip portion may be deformable in response to an external pressure applied to the outer wall of the tip portion.

### CROSS-REFERENCE TO RELATED APPLICATIONS

This application claims priority from U.S. provisional application No. 61/786,866, filed on Mar. 15, 2013, the entirety of which is hereby fully incorporated by reference herein.

### TECHNICAL FIELD

This disclosure relates generally to medical devices. More specifically, this disclosure relates to pressure sensing catheters for detecting the pressure within a body cavity.

### BACKGROUND

The pressure within a body cavity is an important physiological measurement that may be useful in diagnosing and/or treating a variety of medical conditions. Typically, such a pressure measurement is performed using a medical device including a pressure sensor that is coupled to a tube such as an indwelling catheter. An open end of the tube is exposed to the body cavity so that the lumen of the tube is in fluid communication with the body cavity and exposed to the pressure within the body cavity. The pressure within the tube is measured as an indication of the pressure within the body cavity. The tube may be filled with a static fluid (e.g., water or air) to transmit the pressure at the open end of the tube to the pressure sensor at the opposite end of the tube so that it is not necessary for the length of the tube to be filled with the body fluid from the body cavity.

Water charged catheters are sensitive to movement and/or changes in the position of the tube. Therefore, water charged catheters generally must be zeroed or leveled before use to account for the weight of the water positioned above the pressure sensor. Generally, water charged catheters also must be cleared of air bubbles or kinks, which may degrade the pressure measurement of the device. Air charged catheters generally are less sensitive to movement and/or changes in position of the tube than water charged catheters.

Another medical device that may be used for performing a pressure measurement within a body cavity includes a pressure sensor mounted on the exterior of a catheter. Typically, the pressure sensor is mounted on the distal end of the catheter and positioned within the body cavity to measure the pressure within the body cavity directly. Such devices generally are not affected by movement or changes in position of the catheter, as the pressure sensor is positioned within the body cavity. Because the pressure sensor is placed within the body cavity, the pressure sensor must be cleaned before and after each use.

### SUMMARY

The present embodiments provide a pressure sensing catheter for placement within a body cavity to detect the pressure within the body cavity.

In one example, a pressure sensing catheter may include an elongate tubular catheter body having a proximal end, a distal end, a first lumen extending longitudinally within the catheter body, and a second lumen extending longitudinally within the catheter body adjacent to the first lumen. Each of the first lumen and the second lumen may include a proximal end opening positioned at the proximal end of the catheter body and a distal end opening positioned at the distal end of the catheter body. A tip portion may extend distally from the distal end of the catheter body. The tip portion may include an outer wall and a tip lumen within the outer wall. The tip lumen may include a first end opening coupled to the distal end opening of the first lumen and a second end opening adjacent to the first end opening and coupled to the distal end opening of the second lumen. The first lumen and the second lumen may be in fluid communication with one another through the tip lumen. The tip portion may be deformable in response to an external pressure applied to the outer wall of the tip portion.

In another example, a pressure sensing catheter may include a multi-lumen tubular catheter body including an outer wall, a proximal end, a distal end, a first lumen extending longitudinally within the outer wall of the catheter body, and a second lumen adjacent to the first lumen and extending longitudinally within the outer wall of the catheter body. Each of the first lumen and the second lumen may include a proximal end opening positioned at the proximal end of the catheter body and a distal end opening positioned at the distal end of the catheter body. A tip portion may be positioned distal of the distal end of the catheter body. The tip portion may include an outer wall and a tip lumen within the outer wall. The tip lumen may include a first end opening fluidly coupled to the distal end opening of the first lumen and a second end opening fluidly coupled to the distal end opening of the second lumen. The first lumen and the second lumen may be in fluid communication with one another through the tip lumen. The tip portion may be responsive to an external pressure applied to the outer wall of the tip portion.

In another example, a method for determining an external pressure exerted on a pressure sensing catheter may include supplying a fluid to the catheter. The catheter may include a catheter body and a tip portion extending distally from the catheter body. The fluid may flow distally through a first lumen within the catheter body to the tip portion, through a tip lumen within the tip portion, and proximally through a second lumen within the catheter body adjacent to the first lumen. The external pressure exerted on the tip portion may be determined based on a pressure of the fluid supplied to the catheter and a flow rate of the fluid through the catheter.

Other systems, methods, features, and advantages of the invention will be, or will become, apparent to one with skill in the art upon examination of the following figures and detailed description. It is intended that all such additional systems, methods, features, and advantages be within the scope of the invention, and be encompassed by the following claims.

### BRIEF DESCRIPTIONS OF THE DRAWINGS

FIG. 1 illustrates one example of a pressure sensing catheter.

FIG. 2 illustrates a longitudinal cross sectional view of one example of a pressure sensing catheter having a tapered tip portion.

FIG. 3 illustrates a schematic view of one example of a pressure sensing system including a fluid supply system coupled to a pressure sensing catheter.

FIGS. 4-6 illustrate transverse cross sectional views of different examples of multi-lumen tubing.

FIG. 7 illustrates a longitudinal cross sectional view of another example of a pressure sensing catheter having a tip portion with an extension segment and an end segment.

FIG. 8 illustrates a longitudinal cross sectional view of another example of a pressure sensing catheter having a tip portion configured as a U-shaped tube.

FIG. 9 illustrates a longitudinal cross sectional view of another example of a pressure sensing catheter having a tip portion configured as a balloon.

### DETAILED DESCRIPTION OF THE DRAWINGS AND THE PRESENTLY PREFERRED EMBODIMENTS

Detailed embodiments of the present invention are disclosed herein. It is understood, however, that the disclosed embodiments are merely exemplary of the invention, which may be embodied in various and alternative forms. The figures are not necessarily to scale, and some figures may be configured to show the details of a particular component. Therefore, specific structural and functional details disclosed herein are not to be interpreted as limiting, but merely as a representative basis for the claims and for teaching one skilled in the art to practice the present invention.

In the present disclosure, the term “proximal” refers to a direction that is generally toward a physician during a medical procedure, while the term “distal” refers to a direction that is generally toward a target site within a patient's anatomy during a medical procedure.

Various medical devices for introduction within a body cavity are disclosed herein. Preferred embodiments relate to a pressure sensing catheter for placement within a body cavity to detect the pressure within the body cavity. The body cavity may include any space within a body. For example, the body cavity may include a body vessel (e.g., a blood vessel such as an artery or a vein), a bladder, a kidney, an abscess, a bile duct, a ureteropelvic junction, or any other space within a body.

FIG. 1 illustrates one example of a pressure sensing catheter 100. The pressure sensing catheter 100 may be part of a pressure sensing system, which may include a fluid supply system 200 coupled to the pressure sensing catheter as shown in FIG. 3 and further described below. Returning to FIG. 1, the pressure sensing catheter 100 may include a catheter body 110, which may be configured as an elongate tubular member. The catheter body 110 may include a proximal end 112 and a distal end 114. A tip portion 116 may be disposed at the distal end 114 of the catheter body 110. The tip portion 116 may extend distally from the distal end 114 of the catheter body 110. The tip portion 116 may have a tapered outer surface which may aid in introducing the distal end 114 of the catheter body 110 into a body cavity as further described below. A plurality of lumens may extend longitudinally within the catheter body 110. To that end, the catheter body 110 may be formed from a length of multi-lumen tubing as further described below.

FIG. 2 shows a longitudinal cross sectional view of the catheter body 110 and the tip portion 116 as shown in FIG. 1. The plurality of lumens of the catheter body 110 may include a first, inflow lumen 118 and a second, outflow lumen 120. The inflow lumen 118 and the outflow lumen 120 may be positioned adjacent to one another within the catheter body 110 as shown in FIG. 2. The inflow lumen 118 and the outflow lumen 120 may be positioned in a side-by-side relationship such that the inflow lumen and the outflow lumen are not coaxial with one another. Additionally, or alternatively, the inflow lumen 118 and the outflow lumen 120 may be separated from one another by an inner wall or septum within the catheter body 110. In other examples, the inflow lumen 118 and the outflow lumen may be coaxial such that one of the inflow lumen or the outflow lumen is positioned within the other of the inflow lumen and the outflow lumen. The inflow lumen 118 may extend longitudinally between a proximal end opening 122 and a distal end opening 124. Similarly, the outflow lumen may extend longitudinally between a proximal end opening 126 and a distal end opening 128. In one example, the inflow lumen 118 and the outflow lumen 120 may be substantially identical to one another. In other words, the inflow lumen 118 and the outflow lumen 120 may have substantially identical dimensions (e.g., diameter and/or length) and/or substantially identical shapes. In other examples, the inflow lumen and the outflow lumen may have different dimensions and/or shapes.

The tip portion 116 may include a tip lumen 130, which may be in fluid communication with each of the inflow lumen 118 and the outflow lumen 120. The tip lumen 130 may extend between a first end opening 132 and a second end opening 134. The first end opening 132 and the second end opening 134 may be positioned adjacent to one another as shown in FIG. 2. The first end opening 132 of the tip lumen may be fluidly coupled to the distal end opening 124 of the inflow lumen 118. Similarly, the second end opening 134 of the tip lumen 130 may be fluidly coupled to the distal end opening 128 of the outflow lumen 120. In this manner, the inflow lumen 118 and the outflow lumen 120 may be in fluid communication with one another via the tip lumen 130. A continuous fluid pathway may be formed from the proximal end opening 122 of the inflow lumen 118, proximally through the inflow lumen, through the tip lumen 130, and distally through the outflow lumen 120 to the proximal end opening 126 of the outflow lumen. A fluid flow may be established through the continuous fluid pathway to detect an external pressure at the distal tip 116 as further described below. Positioning the first end opening 132 and the second end opening 134 of the tip lumen 130 adjacent to one another may aid in establishing a substantially smooth fluid flow through the tip lumen. For example, turbulence that may be caused within the tip lumen by portion of the fluid flowing in different directions may be reduced by positioning the first end opening 132 and the second end opening 134 adjacent to one another (e.g., because substantially all of the fluid may be directed along a path from the first end opening to the second end opening).

In one example, the tip portion 116 may be formed at the distal end 114 of the catheter body 110 using a tipping process. For example, the distal end 114 of the catheter body 110 may be placed within a tip mold. The tip mold may have an inner surface with a shape that is complementary to the outer surface of the tip portion 116 (e.g., the tapered outer surface described above). Heat and/or pressure may be applied to the distal end 114 of the catheter body 110 to form the outer wall of the tip portion 116. The distal end of the tip portion 116 may be closed or occluded as shown in FIG. 2 so that there is no leakage of fluid from within the tip lumen 130. This may aid in maintaining a closed system as further described below. Additionally, or alternatively, additional material may be added to the tip mold during the tipping process to form the tip portion 116. The additional material may be the same or different than the material used to form the catheter body 110. For example, the additional material may have a lower durometer than the material used to form the catheter body so that the tip portion is softer than the catheter body. During the tipping process, the inflow lumen 118 and the outflow lumen 120 may combine into a single cavity in the tipped junction (e.g., the tip lumen 130).

Returning to FIG. 1, the pressure sensing catheter 100 may include a manifold 140 disposed at the proximal end 112 of the catheter body 110. The manifold 140 may be configured to fluidly couple one or more of the lumens of the catheter body 110 to one or more extension tubes. For example, the manifold 140 may be configured to fluidly couple the inflow lumen 118 to an inflow extension tube 142 and/or to fluidly couple the outflow lumen 120 to an outflow extension tube 144 as shown in FIG. 1. To that end, the manifold 140 may include an inflow channel in fluid communication with each of the inflow lumen 118 and the inflow extension tube 142 and an outflow channel in fluid communication with each of the outflow lumen 120 and the outflow extension tube 144. The manifold 140 may have any suitable configuration known in the art. Additionally, or alternatively, the manifold 140 may be joined to the catheter body 110 by any suitable method including, for example, insert molding the proximal end 112 of the catheter body in the manifold.

The inflow extension tube 142 may be in fluid communication with the inflow lumen 118 of the catheter body 110 through the manifold 140 as described above. An inflow connector 146 may be disposed at a proximal end of the inflow extension tube 142. The inflow connector 146 may be engaged with a corresponding connector of the fluid supply system 200 to supply fluid to the inflow lumen 118 of the pressure sensing catheter 100 as further described below. The outflow extension tube 144 may be in fluid communication with the outflow lumen 120 of the catheter body 110 through the manifold 140 as described above. An outflow connector 148 may be disposed at a proximal end of the outflow extension tube 144. The outflow connector 148 may be engaged with a corresponding connector of the fluid supply system 200 to return fluid from the outflow lumen 120 of the pressure sensing catheter 100 as further described below. The inflow connector 146 and/or the outflow connector 148 may be configured as any suitable connector known in the art such as, for example, a Luer lock. A clamp 150 may be positioned on the inflow extension tube 142 and/or the outflow extension tube 144. The clamp 150 may be any type of conventional clamp configured to prevent fluid flow within the respective extension tube. The clamps 150 may be used to isolate the catheter body 110 and the fluid supply system 200 from one another (e.g., when the fluid supply system is not in operation).

FIG. 3 is a schematic representation of a pressure sensing system including the pressure sensing catheter 100 operatively coupled to the fluid supply system 200. The fluid supply system may include a reservoir 210 configured to hold a fluid 212. In one example, the fluid 212 may be a saline solution. In other examples, the fluid may be any other suitable fluid. The pressure sensing catheter 100 may be in fluid communication with the reservoir 210 via one or more fluid lines. For example, a fluid supply line 214 may be coupled to the reservoir 210 and the pressure sensing catheter 100 (e.g., the inflow extension tube 142) to supply the fluid 212 to the catheter. Additionally, or alternatively, a fluid return line 216 may be coupled to the pressure sensing catheter 100 (e.g., the outflow extension tube 144) and the reservoir 210 to return the fluid 212 to the reservoir. Each of the fluid supply line 214 and the fluid return line 216 may include a unitary tubular segment or a plurality of tubular segments fluidly coupled to one another. In any of the examples described herein, the fluid line may be configured as any type of tubular member configured to enable fluid to flow through the fluid line. For example, the fluid line may include a length of tubing, pipe, or any other type of tubular conduit.

The fluid supply system 200 may include a pump 220. The pump 220 may be configured to pump or motivate the fluid 212 to flow from the reservoir 210, through the fluid supply line 214, to the pressure sensing catheter 100. Additionally, or alternatively, the pump 220 may be configured to pump the fluid 212 through the pressure sensing catheter 100 as further described below and back to the reservoir 210. To that end, the pump 220 may be configured as any type of pump known in the art such as, for example, a peristaltic pump, a gear pump, a vane pump, a centrifugal pump, a positive displacement pump, or any other type of pump. An inlet of the pump 220 may be fluidly coupled to the reservoir 210 (e.g., via the fluid supply line 214). An outlet of the pump may be fluidly coupled to the pressure sensing catheter 100 (e.g., via the fluid supply line 214). The pump 220 may be configured to supply the fluid 212 at a substantially constant flow or at a substantially constant pressure as further described below.

The fluid supply system 200 may include a pressure sensor 222. The pressure sensor 222 may be configured to measure a pressure of the fluid 212 within a fluid line. For example, the pressure sensor may be coupled to the fluid supply line 214 (e.g., downstream of the pump 220) to measure the pressure of the fluid 212 supplied to the pressure sensing catheter 100. The pressure sensor 222 may be configured as a piezoresistive sensor, a capacitive sensor, an electromagnetic sensor, a piezoelectric sensor, an optical sensor, a potentiometric sensor, a resonant sensor, a thermal sensor, an ionization sensor, or any other suitable pressure sensing device.

The fluid supply system 200 may include a flow sensor 224. The flow sensor 224 may be configured to measure a flow rate of the fluid 212 within a fluid line. For example, the flow sensor may be coupled to the fluid supply line 214 to measure the flow rate of the fluid 212 supplied to the pressure sensing catheter 100. Additionally, or alternatively, the flow sensor may be coupled to the fluid return line 216 to measure the flow rate of the fluid 212 returned to the reservoir 210. In one example, the fluid flow path between the reservoir 210 and the pressure sensing catheter 100 may be a closed system such that the flow rate of the fluid 212 supplied to the catheter is equal to the flow rate returned to the reservoir as further described below. The flow sensor 224 may be configured as a piston meter, a gear meter, a nutating disk meter, a variable area flow meter, a turbine flow meter, a Woltmann meter, a single jet meter, a paddle wheel meter, a multiple jet meter, a Pelton wheel, a current meter, a Venturi meter, an orifice plate, a Dall tube, a pitot tube, a multi-hole pressure probe, an optical flow meter, a thermal mass flow meter, a vortex flow meter, a magnetic flow meter, an ultrasonic flow meter, a Coriolis flow meter, or any other suitable flow sensing device.

The fluid supply system 200 may include a control system 226. The control system 226 may be operatively coupled to the pump 220, the pressure sensor 222, and/or the flow sensor 224. For example, the control system 226 may be configured to receive a pressure signal from the pressure sensor 222 and/or a flow signal from the flow sensor 224. Additionally, or alternatively, the control system 226 may be configured to send a control signal to the pump 220. In one example, the control system 226 may be configured to control and/or adjust operation of the pump 220 in response to the pressure signal to maintain a constant pressure in the fluid supply line 214. In one example, the control system 226 may be configured to control and/or adjust the operation of the pump 220 in response to the flow signal to maintain a constant flow rate in the fluid supply line 214 and/or the fluid return line 216. This may enable detection of an external pressure exerted on the pressure sensing catheter 100 as further described below. Additionally, or alternatively, the control system 226 may be configured to calculate the external pressure applied to the pressure sensing catheter 100 and/or changes in the external pressure exerted on the catheter based on the pressure signal received from the pressure sensor 222 and/or the flow signal received from the flow sensor 224 as further described below.

Operation of the pressure sensing system to detect a pressure within a body cavity will be described below with reference to FIGS. 1-3. The pressure sensing catheter 100 may be introduced into a patient's body in any suitable manner. In one example, the tip portion 116 may be introduced into a body vessel, and the pressure sensing catheter 100 may be advanced within the body vessel until the tip portion is positioned within a body cavity in which the pressure is to be measured. In another example, the tip portion 116 may be introduced through a body tissue (e.g., percutaneously), and the pressure sensing catheter 100 may be advanced until the tip portion is positioned within the body cavity in which the pressure is to be measured. Once in position within the body cavity, an external pressure may be exerted on the pressure sensing catheter 100. For example, a fluid pressure may be exerted on the tip portion 116 by the body fluid within the body cavity.

A fluid flow may be established within the pressure sensing system. To that end, the pump 220 may be activated to draw the fluid 212 from the reservoir 210 into the fluid supply line 214 and through the pump. The fluid 212 may flow through the pressure sensor 222 and/or the flow sensor 224. The inflow extension tube 142 may be coupled to the fluid supply line 214 such that the fluid 212 may flow through the fluid supply line into the inflow extension tube. The fluid may flow through the manifold 140 and into the inflow lumen 118 of the catheter body 110 via the proximal end opening 122. The fluid 212 may flow distally within the inflow lumen 118 to the distal end opening 124. The fluid 212 may flow into the tip lumen 130 via the first end opening 132, through the tip lumen, and out of the tip lumen via the second end opening 134. The fluid 212 may flow into the outflow lumen 120 of the catheter body 110 via the distal end opening 128. The fluid 212 may flow proximally within the outflow lumen 120 to the proximal end opening 126. The fluid 212 may flow through the manifold 140 and into the outflow extension tube 142. The outflow extension tube 142 may be coupled to the fluid return line 216 of the fluid supply system 200 such that the fluid 212 may flow through the fluid return line into the reservoir 210. In this manner, the fluid 212 may be caused to flow in a fluid flow path from the reservoir 210, through the catheter body 110 and the tip portion 116, and back to the reservoir. The fluid 212 may be substantially unable to exit the fluid flow path such that substantially all of the fluid that is drawn out of the reservoir 210 via the fluid supply line 214 is returned to the reservoir via the fluid return line 216. In other words, the pressure sensing system may be configured as a closed system. This may aid in maintaining the system in a sterile manner. Additionally, or alternatively, this may aid in preventing the fluid 212 from being introduced into the patient (e.g., into the body cavity in which the pressure is to be measured).

A portion of the pressure sensing catheter 100 (e.g., a portion of the catheter body 110 and/or the tip portion 116) may be configured to be responsive to an external pressure applied to the catheter (e.g., the pressure exerted on the portion of the catheter positioned within the body cavity). In one example, the responsive portion of the pressure sensing catheter 100 may include a relatively thin outer wall. Additionally, or alternatively, the responsive portion may have a relatively low stiffness or hardness. For example, the responsive portion may be formed from a relatively compliant or soft material.

In one example, the tip portion 116 of the pressure sensing catheter 100 may be configured to be responsive to the external pressure applied to the catheter. In other words, the tip portion 116 may be configured as a measuring tip for measuring the external pressure applied to the tip portion. The position of the tip portion within the body cavity may be precisely controlled (e.g., by advancing and/or retracting the catheter). In this manner, measuring the pressure exerted on the tip portion may enable the measurement to be taken at a precise position within the body cavity. At least a portion of the outer wall of the tip portion 116 adjacent to the tip lumen 130 may have a thickness that is less than a thickness of the outer wall of the catheter body 110. In other words, at least a portion of the outer wall surrounding the tip lumen 130 may be thinner than the outer wall of the catheter body 110. Additionally, or alternatively, at least a portion of the outer wall of the tip portion 116 adjacent to the tip lumen 130 may have a stiffness or durometer that is less than a stiffness or durometer of the catheter body 110. In other words, at least a portion of the outer wall surrounding the tip lumen 130 may be more compliant or softer than the outer wall of the catheter body 110.

As the external pressure applied to the outer wall of the pressure sensing catheter 100 increases, the outer wall of the tip portion 116 may deform (e.g., bend or flex inward). In other words, the tip portion 116 may compress in response to an increase in the external pressure. Conversely, as the external pressure applied to the outer wall of the pressure sensing catheter 100 decreases, the outer wall of the tip portion 116 may deform (e.g., bend or flex outward). In other words, the tip portion 116 may expand in response to a decrease in the external pressure. Such deformation of the tip portion 116 (e.g., compression or expansion) may cause the size and/or shape of the tip lumen 130 to change. For example, the tip lumen 130 may become smaller in response to an increase in the external pressure applied to the outer wall of the tip portion 116. Conversely, the tip lumen 130 may become larger in response to a decrease in the external pressure applied to the outer wall of the tip portion 116.

Changes in the size and/or shape of the tip lumen 130 may affect the flow properties of the fluid 212 flowing through the pressure sensing system. For example, a decrease in the size (e.g., diameter) of the tip lumen 130 may increase the pressure drop experienced by the fluid 212 flowing through the tip lumen. In other words, the smaller tip lumen 130 may exert an increased resistance to the flow of the fluid 212 through the tip lumen. Conversely, an increase in the size (e.g., diameter) of the tip lumen 130 may decrease the pressure drop experienced by the fluid 212 flowing through the tip lumen. In other words, the larger tip lumen 130 may exert a decreased resistance to the flow of the fluid 212 through the tip lumen.

In one example, the fluid supply system 200 may be configured to maintain a substantially constant flow rate of the fluid 212. For example, the pump 220 may be configured to supply a substantially constant flow rate of the fluid 212 regardless of the pressure within the system over the pressure range of interest. In other words, the pump 220 may be configured as a constant flow pump. The pressure of the fluid 212, which may be detected by the pressure sensor 222, may change in response to a change in the resistance to the flow through the tip lumen 130. For example, the pressure detected by the pressure sensor 222 may increase in response to an increase in the pressure drop within the tip lumen 130. In other words, the pressure of the fluid 212 in the fluid supply line 214 may increase in response to the increased resistance to flow to maintain the substantially constant flow rate. Conversely, the pressure detected by the pressure sensor 222 may decrease in response to a decrease in the pressure drop within the tip lumen 130. In other words, the pressure of the fluid 212 in the fluid supply line 214 may decrease in response to the decreased resistance to flow to maintain the substantially constant flow rate. The pressure detected by the pressure sensor 222 may be used to determine the external pressure exerted on the tip portion 116 of the pressure sensing catheter 100. For example, the pressure detected by the pressure sensor 222 may be a function of (e.g., proportional to) the external pressure exerted on the tip portion 116 such that the detected pressure may be correlated to a corresponding external pressure. Additionally, or alternatively, changes in the detected pressure may be used to detect changes in the external pressure exerted on the tip portion 116. It should be noted that, when the pump 220 is configured as a constant flow pump, the flow sensor 224 may be omitted.

In another example, the control system 226 may receive the flow signal indicative of the flow rate of the fluid 212 from the flow sensor 224. The control system 226 may adjust the pump 220 to maintain a substantially constant flow rate of the fluid 212. For example, the control system 226 may increase the speed of the pump 220 in response to a detected decrease in the flow rate of the fluid 212 and/or decrease the speed of the pump in response to a detected increase in the flow rate of the fluid. In this manner, the control system 226 may be configured to maintain a substantially constant flow rate of the fluid 212. The pressure detected by the pressure sensor 222 may be used to determine the external pressure exerted on the tip portion 116 of the pressure sensing catheter 100 as described above. Additionally, or alternatively, the control system 226 may be configured to maintain the substantially constant flow rate of the fluid 212 in any other suitable manner such as, for example, adjusting a control valve.

In one example, the fluid supply system 200 may be configured to maintain a substantially constant pressure within the fluid supply line 214. For example, the control system 226 may receive the pressure signal indicative of the pressure of the fluid 212 in the fluid supply line 214 from the pressure sensor 222. The control system 226 may adjust the pump 220 to maintain a substantially constant pressure of the fluid 212. For example, the control system 226 may increase the speed of the pump 220 in response to a detected decrease in the pressure of the fluid 212 and/or decrease the speed of the pump in response to a detected increase in the pressure of the fluid. In this manner, the control system 226 may be configured to maintain a substantially constant pressure of the fluid 212 in the fluid supply line 214. The flow rate of the fluid 212, which may be detected by the flow sensor 224, may change in response to a change in the resistance to flow through the tip lumen 130. For example, the flow rate detected by the flow sensor 224 may decrease in response to an increase in the pressure drop within the tip lumen 130. In other words, the flow rate of the fluid 212 may decrease in response to the increased resistance to flow to maintain the substantially constant pressure. Conversely, the flow rate detected by the flow sensor 224 may increase in response to a decrease in the pressure drop within the tip lumen 130. In other words, the flow rate of the fluid 212 may increase in response to the decreased resistance to flow to maintain the substantially constant pressure. The flow rate detected by the flow sensor 224 may be used to determine the external pressure exerted on the tip portion 116 of the pressure sensing catheter 100. For example, the flow rate detected by the flow sensor 224 may be a function of (e.g., proportional to) the external pressure exerted on the tip portion 116 such that the detected flow rate may be correlated to a corresponding external pressure. Additionally, or alternatively, changes in the detected flow rate may be used to detect changes in the external pressure exerted on the tip portion 116.

In another example, the pump 220 may be configured to supply the fluid 212 at a substantially constant pressure regardless of the flow rate of the fluid 212 over the flow rate range of interest. In other words, the pump 220 may be configured as a constant pressure pump. The flow rate detected by the flow sensor 224 may be used to determine the external pressure exerted on the tip portion 116 of the pressure sensing catheter 100 as described above.

The catheter body 110 may be configured as a length of multi-lumen tubing. FIG. 4 shows a transverse cross sectional view of one example of a multi-lumen tubing. The inflow lumen 118 and the outflow lumen 120 may be configured as substantially cylindrical lumens positioned adjacent to one another and extending longitudinally within the tubing. In other words, each of the inflow lumen 118 and the outflow lumen 120 may have a substantially circular cross sectional shape as shown in FIG. 4. In another example, each of the inflow lumen 118 and the outflow lumen 120 may have a D-shaped cross section as shown in FIG. 5. The curved edge of each D-shaped lumen may substantially conform to the outer curvature of the tubing. In this manner, the cross sectional area of the solid portion of the tubing (e.g., the interior wall or septum) may be reduced and/or the cross sectional area of the lumens may be increased relative to the example shown in FIG. 4. This may aid in reducing the pressure drop experienced by the fluid 212 flowing through the catheter body 110 as described above. In other examples, the inflow lumen 118 and/or the outflow lumen 120 may have any other suitable shape. Additionally, or alternatively, the inflow lumen 118 and the outflow lumen 120 may have the same or different shapes.

FIG. 6 shows a transverse cross sectional view of another example of a multi-lumen tubing including a third, guide wire lumen 121. The guide wire lumen 121 may be configured to receive a guide wire in a conventional manner to guide the pressure sensing catheter 110 to a target location within the patient's body. The inflow lumen 118 and the outflow lumen 120 may be adjacent to the guide wire lumen 121 within the tubing. The guide wire lumen 121 may be offset from the center of the tubing in a first direction. Additionally, or alternatively, each of the inflow lumen 118 and the outflow lumen 120 may be offset from the center of the tubing in a second direction opposite the first direction. In this manner, the guide wire received within the guide wire lumen 121 may be spaced from the inflow lumen 118 and the outflow lumen 120 by a sufficient distance to avoid interference with the tip portion 116 coupled to the inflow lumen and the outflow lumen. Additionally, or alternatively, the guide wire lumen may be fluidly isolated from (i.e., not in fluid communication with) each of the inflow lumen 118, the outflow lumen 120, and the tip lumen 130. In this manner, the flow path for the fluid 212 may be closed as described above.

In one example, the tip lumen 130 may be configured as a cavity within the tip portion 116 as shown in FIG. 2. An outer surface of the cavity may be defined by the inner surface of the outer wall of the tip portion 116. In one example, the outer wall of the tip portion 116 may have a substantially uniform thickness along the length and the circumference of the tip portion such that the outer surface of the cavity has a shape substantially corresponding to the tapered outer surface of the tip portion. In other words, the outer surface of the cavity may have a substantially frustoconical shape as shown in FIG. 2. In one example, the outer wall of the tip portion 116 may become progressively thinner in a proximal to distal direction. This may aid in enhancing the responsiveness of the tip portion 116 to the external pressure exerted on the tip portion. In other examples, the thickness of the outer wall of the tip portion 116 may change in a longitudinal and/or circumferential direction such that the cavity may have any other suitable shape.

Additionally, or alternatively, the tip portion 116 may include an interior wall 136, which may be disposed within the cavity as shown in FIG. 2. The cavity may at least partially surround the interior wall 136. The interior wall 136 may be disposed at the distal end 114 of the catheter body 110. For example, the interior wall 136 may be configured as an extension of the portion of the catheter body 110 positioned between the inflow lumen 118 and the outflow lumen 120 (e.g., the interior wall or septum of the catheter body). The width or diameter of the interior wall 136 may decrease in a proximal to distal longitudinal direction. In other words, the interior wall may be tapered in a proximal to distal longitudinal direction. The interior wall 136 may aid in providing a smooth transition for the fluid 212 flowing from the inflow lumen 118 into the cavity and/or for the fluid flowing from the cavity into the outflow lumen 120. This may help to reduce the turbulence of the fluid flowing through the cavity, which may aid in more accurately detecting the external pressure as described above.

Additionally, or alternatively, the tip portion may be at least partially filled. For example, the distal end of the tip portion 116 may be at least partially filled such that the cavity is longitudinally shorter than the tip portion. In other words, the narrow distal end (e.g., the closed distal tip) of the tip portion may be at least partially filled to form a truncated cavity. This may aid in providing a substantially smooth flow path for the fluid 212 flowing through the cavity (e.g., by preventing turbulence that may result from the fluid reaching the narrow distal end of the tip portion).

In one example, the tip portion 116 of the pressure sensing catheter 100 may include an extension segment 116A and an end segment 116B disposed at a distal end of the extension segment as shown in FIG. 7. The extension segment 116A may be configured as a tubular member with a configuration similar to the catheter body 110. Additionally, or alternatively, the extension segment 116A may have an outer diameter that is substantially the same as the outer diameter of the catheter body 110. This may aid in providing a smooth transition between the tip portion 116 and the catheter body 110. The extension segment 116A may extend from the distal end 114 of the catheter body 110. The tip portion 116 may terminate at the end segment 116B, which may be configured as a rounded distal end of the tip portion. The end segment 116B may provide an atraumatic surface for placement of the pressure sensing catheter within the body cavity. The outer surface of the tip portion 116 may have a substantially cylindrical shape along the extension segment 116A as shown in FIG. 7. The outer wall of the extension segment 116A and/or the end segment 116B may be thinner and/or softer than the outer wall of the catheter body 110 as described above. In one example, the tip portion 116 may be formed from a softer material than the catheter body 110. In other words, the catheter body 110 may be formed from a heavier durometer material than the tip portion 116. The catheter body 110 and the tip portion 116 may be coextruded such that the relatively softer tip portion extends from the distal end 114 of the catheter body. In another example, the catheter body 110 and the tip portion 116 may be formed from distinct lengths of multi-lumen tubing joined to one another such that the tip portion extends from the distal end 114 of the catheter body. The distal end of the tip portion 116 may be closed (e.g., using a molding or tipping process) to form the end segment 116B.

The tip lumen 130 may be configured as a U-shaped lumen disposed within the tip portion 116 as shown in FIG. 7. The U-shaped lumen may include two substantially parallel legs extending longitudinally within the extension segment 116A and coupled to one another by a bend. The legs may be separated from one another by a solid portion of the tip portion 116 (e.g., the interior wall 136 between adjacent lumens of the multi-lumen tubing). The bend may be configured as a C-shaped segment of the tip lumen 130 positioned within the end segment 116B. The first leg may extend distally from the first end opening 132 to the bend. The second leg may extend proximally from the bend to the second end opening 134.

Upon introduction of the fluid 212 into the tip lumen 130, the tip portion 116 (e.g., the extension segment 116A and/or the end segment 116B) may swell like a balloon. In other words, the pressure of the fluid 212 flowing through the tip lumen 116 may cause the tip portion 116 to expand radially outward. This may cause the outer wall of the tip portion 116 to stretch, which may reduce the thickness of the outer wall. Such swelling of the tip portion 116 and/or stretching of the outer wall may help to increase the responsiveness of the tip portion to the external pressure exerted on the tip portion as described above.

In one example, the tip portion 116 of the pressure sensing catheter 100 may be configured as a U-shaped tube extending distally from the distal end 114 of the catheter body 110 as shown in FIG. 8. The U-shaped tube may include two substantially parallel tubular legs extending distally from the catheter body 110 and coupled to one another by a tubular bend. A first leg of the U-shaped tube may be attached to the catheter body 110 adjacent to the distal end opening 124 of the inflow lumen 118, and a second leg of the U-shaped tube may be attached to the catheter body adjacent to the distal end opening 128 of the outflow lumen 120. In one example, the U-shaped tube may be formed from a length of tubing arranged in a U-shape and attached to the catheter body 110 as described above. In another example, the U-shaped tube may be formed integrally with the catheter body (e.g., using a molding or tipping process). The outer wall of the U-shaped tube may be thinner than the outer wall of the catheter body 110 as shown in FIG. 8 and described above. Additionally, or alternatively, the outer wall of the U-shaped tube may be formed from a softer material than the outer wall of the catheter body as described above.

In one example, the U-shaped tube and the distal end 114 of the catheter body 110 may collectively define an opening 117 as shown in FIG. 8. In this manner, the tip lumen 130 may be adjacent to the outer wall of the tip portion 116 around substantially the entire circumference of the tip lumen and along substantially the entire length of the tip lumen. For example, the tip portion 116 may be free of an interior wall (e.g., the interior wall 136 described above with reference to FIG. 7). Additionally, or alternatively, one leg of the U-shaped tip lumen 130 may not be positioned between the outer wall of the tip portion 116 and the other leg of the U-shaped tip lumen (e.g., as in the example shown in FIG. 7). Additionally, or alternatively, the thickness of the outer wall of the tip portion 116, or the distance between the tip lumen 130 and the outer surface of the tip portion, may be substantially constant around the circumference of the tip lumen and along the length of the tip lumen as shown in FIG. 8. This may improve the responsiveness of the tip portion 116 to the external pressure. For example, the tip portion 116 may be responsive to forces exerted from any direction around the U-shaped tube. For example, upon placement of the tip portion 116 within the body cavity, a body fluid disposed within the body cavity may be capable of flowing into the opening 117, and a corresponding pressure may be exerted on the portion of the outer wall of the tip portion adjacent to the opening.

In one example, the tip portion 116 of the pressure sensing catheter 100 may be configured as a balloon extending distally from the distal end 114 of the catheter body 110 as shown in FIG. 9. The balloon may have any shape and/or configuration known in the art. The tip lumen 130 may be configured as a chamber defined within the balloon. To that end, the first end opening 132 may be formed in a wall of the balloon, and the second end opening 134 may be formed in the wall of the balloon. The first end opening 132 and the second end opening 134 may be positioned adjacent to one another to align with the inflow lumen 118 and the outflow lumen 120, respectively, as shown in FIG. 9. The balloon may be configured to expand and/or retract in response to the pressure exerted by the fluid 212 within the tip lumen 130 and/or the external pressure as described above.

While various embodiments of the invention have been described, the invention is not to be restricted except in light of the attached claims and their equivalents. Drawings in the figures illustrating various embodiments are not necessarily to scale. Some drawings may have certain details magnified for emphasis, and any different numbers or proportions of parts should not be read as limiting unless so-designated in the present disclosure. Those skilled in the art will appreciate that embodiments not expressly illustrated herein may be practiced within the scope of the present invention, including those features described herein for different embodiments, which may be combined with each other and/or with currently-known or future-developed technologies while remaining within the scope of the claims presented herein. Moreover, the advantages described herein are not necessarily the only advantages of the invention and it is not necessarily expected that every embodiment of the invention will achieve all of the advantages described.

We claim:

1. A pressure sensing catheter comprising:
an elongate tubular catheter body comprising a proximal end, a distal end, a first lumen extending longitudinally within the catheter body, and a second lumen extending longitudinally within the catheter body adjacent to the first lumen, each of the first lumen and the second lumen comprising a proximal end opening positioned at the proximal end of the catheter body and a distal end opening positioned at the distal end of the catheter body; and
a tip portion extending distally from the distal end of the catheter body and comprising an outer wall and a tip lumen within the outer wall, the tip lumen comprising a first end opening coupled to the distal end opening of the first lumen and a second end opening adjacent to the first end opening and coupled to the distal end opening of the second lumen, the first lumen and the second lumen in fluid communication with one another through the tip lumen, the tip portion being deformable in response to an external pressure applied to the outer wall of the tip portion.

2. The catheter of claim 1, wherein the outer wall of the tip portion is thinner than an outer wall of the catheter body.

3. The catheter of claim 1, wherein the outer wall of the tip portion is softer than an outer wall of the catheter body.

4. The catheter of claim 1, wherein the tip portion comprises a U-shaped tubular member extending distally from the distal end of the catheter body, and the tip lumen extends longitudinally within the U-shaped tubular member.

5. The catheter of claim 4, wherein the distal end of the catheter body and the tip portion collectively define an opening therebetween.

6. The catheter of claim 1, wherein the tip portion comprises an elongate tapered member comprising a proximal end coupled to the distal end of the catheter body, a distal end, and an outer diameter that is increasingly smaller in a proximal to distal direction, each of the first end opening and the second end opening of the tip lumen is positioned at the proximal end of the tip portion, and the tip lumen comprises a cavity within the tip portion.

7. The catheter of claim 6, wherein the tip portion comprises an interior wall extending distally from the distal end of the catheter body, and the interior wall is at least partially surrounded by the cavity.

8. The catheter of claim 1, wherein the tip portion comprises an extension segment extending distally from the distal end of the catheter body and an end segment disposed at a distal end of the extension segment.

9. The catheter of claim 8, wherein the tip lumen comprises a U-shaped lumen comprising a first leg and a second leg coupled to one another by a bend, each of the first leg and the second leg extends longitudinally within the extension segment, and the bend is disposed within the end segment.

10. The catheter of claim 1, wherein the catheter body further comprises a third lumen extending longitudinally within the catheter body adjacent to each of the first lumen and the second lumen and configured to receive a guide wire.

11. The catheter of claim 10, wherein each of the first lumen and the second lumen is offset from a center of the catheter body in a first direction, and the third lumen is offset from the center of the catheter body in a second direction opposite the first direction.

12. The catheter of claim 1, wherein the catheter body comprises a length of multi-lumen tubing.

13. The catheter of claim 1, further comprising a manifold positioned at the proximal end of the catheter body, a first extension tube coupled to the manifold and in fluid communication with the first lumen of the catheter body, and a second extension tube coupled to the manifold and in fluid communication with the second lumen of the catheter body.

14. A pressure sensing system comprising the catheter of claim 1, the system further comprising:
a pump operatively coupled to the proximal end opening of the first lumen to supply a fluid to the first lumen, the fluid flowing proximally through the first lumen, through the tip lumen, and distally through the second lumen; and
a pressure sensor to detect a pressure of the fluid supplied to the first lumen.

15. A pressure sensing catheter comprising:
a multi-lumen tubular catheter body comprising an outer wall, a proximal end, a distal end, a first lumen extending longitudinally within the outer wall of the catheter body, and a second lumen adjacent to the first lumen and extending longitudinally within the outer wall of the catheter body, each of the first lumen and the second lumen comprising a proximal end opening positioned at the proximal end of the catheter body and a distal end opening positioned at the distal end of the catheter body; and
a tip portion positioned distal of the distal end of the catheter body and comprising an outer wall and a tip lumen within the outer wall, the tip lumen comprising a first end opening fluidly coupled to the distal end opening of the first lumen and a second end opening fluidly coupled to the distal end opening of the second lumen, the first lumen and the second lumen in fluid communication with one another through the tip lumen, the tip portion being responsive to an external pressure applied to the outer wall of the tip portion.

16. The catheter of claim 15, wherein the tip lumen comprises a U-shaped lumen comprising a first leg and a second leg coupled to one another by a bend.

17. The catheter of claim 16, wherein the tip portion comprises a U-shaped tube comprising a first end attached to the distal end of the catheter body and a second end attached to the distal end of the catheter body adjacent to the first end, and the tip lumen extends longitudinally within the U-shaped tube.

18. The catheter of claim 16, wherein the tip portion comprises a multi-lumen tubular extension segment extending distally from the catheter body and an end segment at a distal end of the extension segment, and the first leg and the second leg are positioned adjacent to one another within the extension segment and spaced from one another by an interior wall.

19. The catheter of claim 15, wherein the outer wall of the tip portion comprises at least one of a lesser thickness or a lesser durometer than the outer wall of the catheter body.

20. A method for determining an external pressure exerted on a pressure sensing catheter, the method comprising:
supplying a fluid to the catheter, the catheter comprising a catheter body and a tip portion extending distally from the catheter body, the fluid flowing distally through a first lumen within the catheter body to the tip portion, through a tip lumen within the tip portion, and proximally through a second lumen within the catheter body adjacent to the first lumen; and
determining the external pressure exerted on the tip portion based on a pressure of the fluid supplied to the catheter and a flow rate of the fluid through the catheter.
